(19) United States
(12) Patent Application Publication
(10) Pub. No.: US 2026/0213018 A1
(43) Pub. Date: Jul. 23, 2026

(54) SYSTEM AND METHOD FOR PREDICTING A HEALTH-RELATED OUTCOME USING INTEGRATION OF HEALTH-RELATED MODALITIES DATA BASED ON A LARGE LANGUAGE MODEL

(71) Applicant: KNOWTIONS RESEARCH INC., Toronto (CA)

(72) Inventors: Hanieh Arjmand, Thornhill (CA); Alexandre TOMBERG, Scarborough (CA)

(21) Appl. No.: 19/198,309

(22) Filed: May 5, 2025

Related U.S. Application Data
(60) Provisional application No. 63/747,204, filed on Jan. 20, 2025.

(51) Int. Cl. G16H 50/30; G16H 10/60

(52) U.S. Cl. CPC G16H 50/30; G16H 10/60

(57) ABSTRACT

A system and method for predicting a health-related outcome. The method including: receiving electronic health records data; generating a first set of latent representations using a first trained encoder, the first trained encoder taking the electronic health records data as input, the first trained encoder trained to extract health patterns from the inputted electronic health records data; generating integrated embeddings by integrating the first latent representations into an integrated embedding space; generating a prediction for the health-related outcome using a trained large language model, the trained large language model taking the integrated embeddings in the integrated embedding space as input, a training dataset for training of the large language model includes electronic health records data and supplementary modality data, integrated embeddings during training of the large language model further includes integrated embeddings of a second set of latent representations generated by a second trained encoder from the supplementary modality data.

### TECHNICAL FIELD

The following relates generally to the field of predictive medicine, and more particularly, to systems and methods for predicting a health-related outcome using integration of health-related modalities data.

### BACKGROUND

Recent advancements in healthcare have led to the accumulation of extensive data from diverse sources and modalities, such as electronic health records (EHR) and wearable devices. EHRs, for example, provide a structured view of patient history, encompassing details such as diagnosis codes, clinical visits, and treatment records. Wearable devices, as another example, offer real-time and historical physiological data such as step counts and heart rate. Other data sources and modalities provide other data related to a person and these data sources are generally independent and traditionally have been analyzed separately.

### SUMMARY

In an aspect, there is provided a computer-implemented method for predicting a health-related outcome, the method comprising: receiving electronic health records data; generating a first set of latent representations using a first trained encoder, the first trained encoder taking the electronic health records data as input, the first trained encoder trained to extract health patterns from the inputted electronic health records data; generating integrated embeddings by integrating the first latent representations into an integrated embedding space; generating a prediction for the health-related outcome using a trained large language model, the trained large language model taking the integrated embeddings in the integrated embedding space as input, a training dataset for training of the large language model comprises electronic health records data with associated labelled health outcomes and supplementary modality data with associated labelled health outcomes, the integrated embeddings during training of the large language model further comprise integrated embeddings of a second set of latent representations generated by a second trained encoder from the supplementary modality data; outputting the prediction for the health-related outcome.

In a particular case of the method, the supplementary modality data comprises time-series wearable activity data.

In another case of the method, the health patterns extracted by the first trained encoder comprises patterns in temporal sequences of diagnosis codes.

In yet another case of the method, the first trained encoder is trained using masked code prediction by predicting randomly masked diagnosis codes based on surrounding diagnosis codes.

In yet another case of the method, the first trained encoder uses bidirectional encoder representations from transformers.

In yet another case of the method, the second trained encoder comprises a one-dimensional convolutional neural network combined with fully connected layers.

In yet another case of the method, the training data for the second trained encoder comprises time-stamped activity data, and wherein during training of the second trained encoder, the second trained encoder is trained to predict mortality by identifying patterns in activity levels linked to health outcomes.

In yet another case of the method, the training dataset further comprises additional supplementary modality data, and wherein the integrated embeddings further comprise integrated embeddings of a third set of latent representations generated by a third trained encoder from the additional supplementary modality data.

In yet another case of the method, the additional supplementary modality data comprises nutritional data, and wherein the third trained encoder comprises a multi-layer perceptron.

In yet another case of the method, the integrated embeddings are generated by performing at least one of encoding alignment, prompt integration, embedding replacement.

In another aspect, there is provided a system for predicting a health-related outcome, the system comprising a processing unit in communication with a data storage, the data storage comprising instructions for the processing unit to execute: an input module to receive electronic health records data; a health records encoding module to generate a first set of latent representations using a first trained encoder, the first trained encoder taking the electronic health records data as input, the first trained encoder trained to extract health patterns from the inputted electronic health records data; an embedding module to generate integrated embeddings by integrating the first latent representations into an integrated embedding space; a prediction module to generate a prediction for the health-related outcome using a trained large language model, the trained large language model taking the integrated embeddings in the integrated embedding space as input, a training dataset for training of the large language model comprises electronic health records data with associated labelled health outcomes and supplementary modality data with associated labelled health outcomes, the integrated embeddings during training of the large language model further comprise integrated embeddings of a second set of latent representations generated by a second trained encoder from the supplementary modality data; an output module to output the prediction for the health-related outcome.

In a particular case of the system, the supplementary modality data comprises time-series wearable activity data.

In another case of the system, the health patterns extracted by the first trained encoder comprises patterns in temporal sequences of diagnosis codes.

In yet another case of the system, the first trained encoder is trained using masked code prediction by predicting randomly masked diagnosis codes based on surrounding diagnosis codes.

In yet another case of the system, the first trained encoder uses bidirectional encoder representations from transformers.

In yet another case of the system, the second trained encoder comprises a one-dimensional convolutional neural network combined with fully connected layers.

In yet another case of the system, the training data for the second trained encoder comprises time-stamped activity data, and wherein during training of the second trained encoder, the second trained encoder is trained to predict mortality by identifying patterns in activity levels linked to health outcomes.

In yet another case of the system, the training dataset further comprises additional supplementary modality data, and wherein the integrated embeddings further comprise integrated embeddings of a third set of latent representations generated by a third trained encoder from the additional supplementary modality data.

In yet another case of the system, the additional supplementary modality data comprises nutritional data, and wherein the third trained encoder comprises a multi-layer perceptron. In yet another case of the system, the integrated embeddings are generated by performing at least one of encoding alignment, prompt integration, embedding replacement.

These and other aspects are contemplated and described herein. It will be appreciated that the foregoing summary sets out representative aspects of systems and methods to assist skilled readers in understanding the following detailed description.

### DESCRIPTION OF THE DRAWINGS

A greater understanding of the embodiments will be had with reference to the Figures, in which:

FIG. 1 is a schematic diagram of a system for predicting a health-related outcome using integration of health-related modalities data, in accordance with an embodiment; and

FIG. 2 is a flowchart of a method for predicting a health-related outcome using integration of health-related modalities data, in accordance with an embodiment.

### DETAILED DESCRIPTION

Embodiments will now be described with reference to the figures. For simplicity and clarity of illustration, reference numerals may be repeated among the Figures to indicate corresponding or analogous elements. Specific details are set forth to provide a thorough understanding of the technical aspects and innovative features of the embodiments described herein. However, it will be understood by those of ordinary skill in the art that the embodiments described herein may be practised without these specific details, provided that the core innovative concepts are retained. In other instances, methods, procedures, and components which are known to a skilled person in the art have not been described in detail so as not to obscure the novel aspects of the embodiments described herein.

Various terms used throughout the present description should be read and understood as follows, unless the context indicates otherwise: “or” is inclusive, as though written “and/or”; singular articles and pronouns include their plural forms, and vice versa; similarly, gendered pronouns include their counterpart pronouns so that pronouns should not be understood as limiting anything described herein to use, implementation, or performance by a single gender; “exemplary” means “illustrative” or “exemplifying” and not necessarily “preferred” over other embodiments. Further definitions for terms are provided to ensure clarity and consistency in understanding the technical features and innovations described herein. These definitions apply to prior and subsequent instances of those terms as understood from reading the present description, ensuring that the novel and inventive aspects are consistently interpreted.

Any module, unit, component, server, computer, terminal, engine, or device exemplified herein that executes instructions described herein, may include access computer readable media, such as storage media, computer storage media, or data storage devices (removable and/or non-removable) such as, for example, magnetic disks, optical disks, or tape. Computer storage media may include volatile and non-volatile, removable, and non-removable media implemented in any method or technology for storing information, such as computer readable instructions, data structures, program modules, or other data. Examples include RAM, ROM, EEPROM, flash memory or other non-transitory memory technology, CD-ROM, digital versatile disks (DVD) or other optical storage, magnetic cassettes, magnetic tape, magnetic disk storage or other magnetic storage devices, or any other non-transitory medium that stores the desired data and/or information and can be accessed for execution of an application, module, or both. Any such non-transitory media may be part of the device or accessible thereto, or may otherwise provide functionality to the system to enable efficient data storage and retrieval. Processors or controllers set out herein may be implemented as a single processor or a plurality of processors, which May be arrayed or distributed, such as in a cloud-based model, in order to carry out processing functions individually or collectively. Methods, applications, or modules described herein may be implemented using computer readable/executable instructions that may be stored or otherwise held by such non-transitory media and executed by one or more processors to be used in the realization of the embodiments describe herein.

The following relates generally to the field of predictive medicine, and more particularly to systems and methods for predicting a health-related outcome using integration of health-related modalities data.

The present embodiments, through the use of advanced models, such as Large Language Models (LLMs), enhance disease prediction for individual patients and have the ability to scale to provide greater insights for public health monitoring and epidemiology. Such models are able to analyze complex, multimodal data to reveal underlying health risks. Particularly, the present embodiments advantageously integrate diverse data modalities into a unified predictive framework to provide substantially better predictive health capabilities. 2 LLMs have conventionally been used in healthcare contexts to generate insights and

personalized recommendations. For example, LLMs have been used to engage users in personalized health conversations, utilizing physiological and contextual data to improve individuals' understanding of their health. In another example, LLMs have been used to interpret wearable health data for responding to health-related questions and develop data-driven wellness strategies.

The integration of EHR and other health related modalities (such as wearable data) for predictive analytics, in the present embodiments, advantageously uses multiple sources for the ingestion and interpretation of complex data to arrive at more complete and accurate predictive analytics. Wearable devices offer continuous, real-time monitoring of physiological parameters, for example, heart rate, activity, and sleep quality. While EHRs provide a longitudinal view of a patient's health, for example, lab results, diagnoses, and clinical notes. Integrating these data streams permits the building of predictive models that can, amongst others, identify at-risk patients early; potentially enabling timely and personalized interventions. Additionally, by combining the continuous flow of real-time wearable data with the depth of historical EHR information, the present embodiments can determine comprehensive health profiles for a particular patient; generally enhancing proactive healthcare approaches that emphasize prevention.

LLM models have the capability to process and synthesize multimodal, individual-specific data. In the context of healthcare data, this can include everything from textual clinical notes to time-series data from wearables; thus, providing a nuanced understanding of a patient's potential health risks. In some cases of the present embodiments, LLMs can be trained or fine-tuned to adapt their responses based on a patient's risk profile, for example, integrating contextual data such as lab values, genetic predispositions, and other indicators. The approach of the present embodiments advantageously leverage extensive prior medical knowledge encoded within LLMs, which can identify connections between conditions, such as recognizing that ‘hypertension’ and ‘high blood pressure’ are synonymous. Additionally, such models can be used to understand clinical significance of various metrics, such as various lab ranges.

Unlike classical predictive models, which often require explicit coding and complex data handling to include prior knowledge, LLMs can utilize prompt engineering techniques to dynamically adjust to new information. This flexibility is in particular useful when dealing with diverse and incomplete datasets; which is common with healthcare-related data. Additionally, LLMs can seamlessly handle gaps in data, focusing on the available information without architectural modifications. In the present embodiments, the present inventors determined that this flexibility adventitiously allows LLMs to make predictions based on heterogeneous datasets that combine real-time wearable data, structured clinical information from EHRs, and in some cases, unstructured text like clinician notes.

Integrating diverse health data types into an LLM model is a substantial and complex challenge. Health data is often stored in varied formats and modalities, from numerical lab results to visual data such as medical imaging. Traditional approaches have addressed this challenge through multimodal fusion techniques, such as logistic regression models that integrate several data inputs or fusion architectures combining features from disparate sources. However, these approaches generally lack the flexibility and scalability of LLMs. For instance, traditional models require cumbersome approaches to incorporate prior knowledge or to deal with fuzzy, non-standardized labels. In the context of LLMs, a significant challenge lies in how to efficiently integrate disparate healthcare data sources into a format that LLMs can process while retaining its richness and clinical relevance. In healthcare, crucial indicators of disease often span multiple data modalities, for example, requiring combining wearable sensor readings with diagnostic information for accurate prediction.

The present invention utilizes capabilities of LLMs via integration of specialized health-related embeddings and retraining of the model within a particular technical framework. This approach provides a multimodal framework that can be used to process EHR embeddings and embeddings from other modalities (such as wearable data) as enriched inputs, enabling the models to derive contextual relationships between clinical history and real-time physiological signals. Advantageously, embodiments of the present disclosure treat the embeddings as newly introduced “words” that enrich the understanding of health-related information in the language model. By embedding these data types into an integrated embedding space, the multimodal framework of the present invention effectively retrains the model to interpret these embeddings with the same familiarity as an existing knowledge base. This allows the present invention to capture complex interactions and nuanced meanings between clinical history and real-time physiological signals, thereby deepening the predictive and contextual capabilities.

Advantageously, the approach of the present embodiments allows the models to “learn” from both structured EHR data and, for example, dynamic activity patterns, similar in effect to how new vocabulary terms expand the capabilities of a language model to understand and contextual relevance. The advantages of the present invention were verified in example experiments, described herein, where trained models were able to improve predictions by combining contextual and physiological data.

In the present embodiments, integration of wearable and EHR data enables improved disease prediction, particularly improving accuracy and allow for the development of customized intervention strategies. For example, LLMs can incorporate patient-specific risk factors, such as genetic history and lifestyle choices, into prompts; thus, facilitating disease prediction and health management, and making the results more accurate and relevant. The advantages of such personalization extend beyond individual care to broader public health implications. Predictive analytics can identify at-risk populations by analyzing aggregated multimodal data, which can inform targeted health interventions and resource allocation. By predicting health risks at both the 14 individual and population level, more effective health policies can be determined and overall quality of care can be enhanced. Additionally, in some cases, the LLMs of the present embodiments can further include a conversational interface, making them ideal for use in health and wellness applications, such as chatbots that offer personalized health advice based on integrated EHR and wearable data.

One of the existing frameworks that leverage a combination of complex clinical data with LLMs is HeLM (Health Large Language Model for Multimodal Understanding); which is used to predict disease risk. HeLM utilizes data from the U.K. Biobank, combining conventional clinical information, such as demographic and laboratory data, with spirogram curves, which are used to evaluate lung function across various diseases. To manage the different data types, HeLM employs modality-specific encoders that convert non-text data into a representation suitable for processing by LLMs. Although the approach of HeLM includes structured tabular data, the tabular format by nature is very limited in scope.

Generally, HeLM uses high-dimensional clinical data and spirogram readings, which are limited in scope to respiratory health and select static variables. The clinical data generally includes lab values (e.g., HDL/LDL cholesterol, triglycerides) and self-reported statuses from questionnaires (e.g., age, sex, BMI, smoking status, insomnia, sleeping patterns, and chronotype). While these inputs provide useful snapshots, they lack the depth and complexity of analyzing EHR data, such as in the present embodiments, which captures rich interactions between variables and temporal sequences of medical events. Moreover, HeLM's inputs do not reflect dynamic physiological changes or longitudinal health patterns comprehensively.

In contrast, the present invention integrates EHR data, providing rich, longitudinal insights, with other modalities, such as wearable-derived time-series data that captures real-time activity trends. This exemplary combination allows for adaptive health predictions across a broader spectrum of conditions, leveraging continuous monitoring and dynamic interactions for nuanced, robust healthcare solutions. Moreover, embodiments of the present invention advantageously leverages EHR for detailed health history and wearable-derived activity data for real-time physiological monitoring, thereby providing a comprehensive and continuous health profile. This integration, which emphasizes proactive disease prediction, addresses challenges associated with combining disparate data modalities, including structured clinical data and continuous time-series data. Such integration of longitudinal EHR data with real-time physiological monitoring is in stark contrast to the HeLM framework, which utilizes high-dimensional clinical data and spirogram readings for disease risk assessment. The integration of longitudinal EHR data with real-time physiological monitoring permits determining adaptive and robust healthcare solutions, which is not possible using the HeLM framework.

Additionally, the present invention uses advantageous encoding strategies, as described herein, to optimize the integration of EHR with other modalities, such as wearable-derived activity data; creating a comprehensive approach to proactive healthcare management. In contrast, HeLM merely uses a Multi-Layer Perceptron (MLP) for tabular data and a ResNet architecture for processing spirogram data.

In a particular embodiment of the present invention, a BERT-based EHR encoder is used for encoding EHR data and a CNN-based encoder is used for encoding the wearable data. Using such encoding enhances the processing of structured clinical information and real-time physiological signals. Combining such encodings in the model bridges the gap between historical health context and real-time monitoring of dynamic physiological changes, offering improved predictive power for a variety of health conditions.

Unlike the HeLM framework, which focuses on respiratory and clinical data interpretation, the present invention provides broader applications which can support adaptive and scalable healthcare solutions. This capability can be used to, for example, capture diverse health-related signals, provide actionable insights for proactive disease management, and/or offer a unique angle on proactive healthcare management, specially as a versatile tool in personalized healthcare.

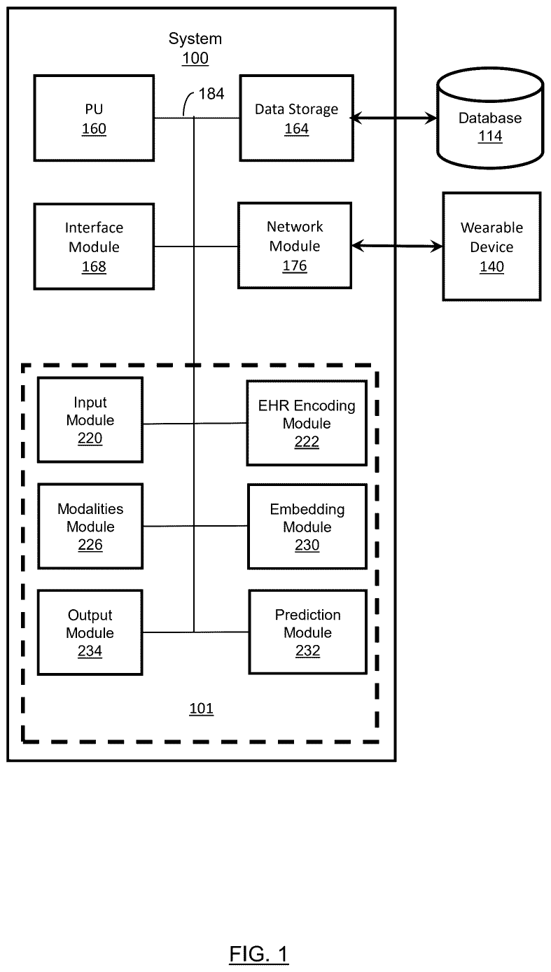

Turning to FIG. 1, shown is a conceptual diagram of a system 100 for predicting a health-related outcome using integration of health-related modalities data, in accordance with an embodiment. As shown, the system 100 has a number of physical and logical components, including a processing unit (“PU”) 160, data storage 164, an interface module 168, a network module 176, and a local bus 184 enabling PU 160 to communicate with the other components. PU 160 can include one or more central processing units, one or more graphical processing unit, microprocessors, dedicated hardware, or other integrated processing circuits. The data storage 164 provides storage of data to the PU 160 and can include volatile and non-volatile storage for storing data required by the PU 160, including computer-executable instructions for implementing the methods described herein, as well as any derivative or related data. In some cases, the data storage 164 can include a database 114, which can be remotely located. The interface module enables input to be provided; for example, directly via a user input device, or indirectly, for example, via an external device. The interface module 168 also enables output to be provided; for example, directly via a user display, or indirectly, for example, sent over the network module 176. The network module 176 permits communication with other systems or computing devices; for example, over a local area network or over the Internet. In some cases, the system 100 can be in communication with one or more wearable devices 140 to send and receive data over a network via the network module 176.

In other embodiments, any operating system, programs, or instructions can be executed in hardware, specialized microprocessors, logic arrays, or the like. While FIG. 1 illustrates a system implemented on a single computing device, it is understood that the processing, or any of the functions undertaken by the system 100, can be distributed over multiple computing devices; for example, in a cloud or distributed computing environment. In this way, the system 100 can run on a standalone, such as on a local computer, or it can share computing resources and/or data located remotely, such as on a server communicating over a network, such as the Internet.

In an embodiment, the PU 160 can be configured to execute a number of conceptual modules 101; for example, input module 220, a health records encoding module 222, a modalities module 226, an embedding module 230, a prediction module 232, and an output module 234. In further cases, functions of the above modules can be combined or executed on other modules. In some cases, functions of the above modules can be executed on remote computing devices, such as centralized servers and cloud computing resources communicating over the network module 176.

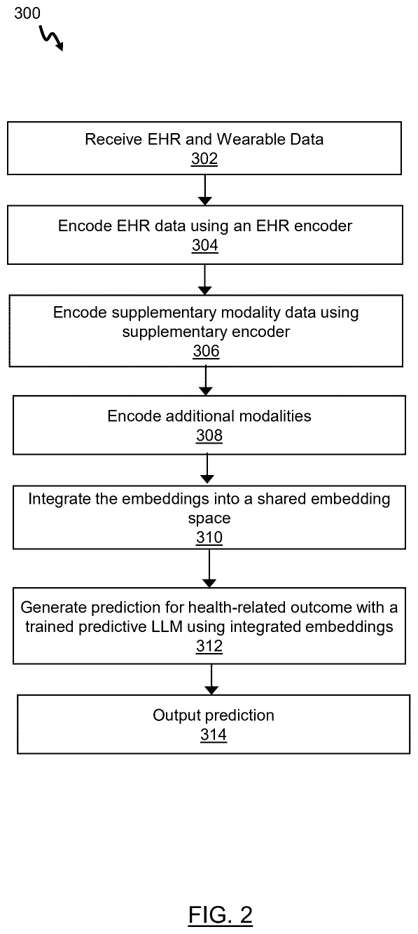

FIG. 2 shows a method 300 for predicting a health-related outcome using integration of health-related modalities data. At block 302, the input module 220 receives EHR data and data of a supplementary modality; for example, via the database 214, the network interface 208 and/or the user interface 206. The received data includes both structured EHR data and supplementary modality data with one or more other modalities, which may be received from different sources. In a particular case, the received data includes time-series wearable activity data. The EHR data can be collected and accumulated from one or more sources, for example, health record databases, hospital records, governmental health repositories, and health insurance related databases. The supplementary modality data can be sourced from suitable sources, which can include the same sources that also store the EHR data. In the case where the supplementary modality data is wearable time-series activity data, such data may be collected directly from wearable devices, or from sources that store the data derived from the wearable devices. Each data source may host the various sources of data in different formats (in some cases, in proprietary formats).

Generally, the EHR data is structured into standardized formats such as International Classification of Diseases (ICD) codes for diagnoses, Current Procedural Terminology (CPT) or Healthcare Common Procedure Coding System (HCPCS) codes for procedures, lab results with predefined units, medication records, and temporal information about patient visits. This structured format enables systematic processing and analysis by the encoders of the present invention to extract meaningful health patterns while preserving temporal relationships between medical events.

At block 304, the health records encoding module 222 encodes the structured EHR data to produce latent representations of the structured EHR input data. The health records encoding module 222 uses an EHR encoder that captures patterns in diagnosis codes within the structured EHR to reflect complex relationships in the clinical EHR data. The encoding condenses the raw input data into lower-dimensional vectors in order to focus on various health indicators. Whereby the latent representations of the EHR input data highlight features that are most predictive of health-related (e.g., disease) outcomes and provide an efficient encoding of essential characteristics of the raw EHR input data. The health records encoding module 222 can independently pre-train the EHR encoder to capture temporal and contextual patterns within diagnosis sequences. In this way, the EHR encoder can effectively model longitudinal EHR data for predictive insights.

Generally, the EHR encoder outputs latent representations in the form of multi-dimensional vectors that represent clinical information from the EHR data. These vectors capture the temporal sequence of diagnosis codes, highlighting relationships between codes and their context within a patient's medical history. By analyzing the order and co-occurrence of diagnoses, the encoder distills complex, high-dimensional data into a simplified, lower-dimensional format. This latent representation preserves the most important features, such as patterns in diagnosis progression and their relative significance, ensuring it is effective for downstream tasks like prediction and classification.

The pre-training of the EHR encoder can use any suitable approach. For example, using masked code prediction by predicting randomly masked diagnosis codes based on surrounding diagnosis codes. Further, for example, using prolonged length-of-stay prediction by assessing whether an incident of prolonged hospital stay (e.g., length of stay >7 days) had ever occurred throughout the patient's entire EHR sequence. The EHR encoder can be pretrained with any suitable bank of EHR data, for example, EHR data from the United Kingdom Biobank. In some cases, the pretraining strategy can reserve one year of data for the target, meaning the EHR encoder is never exposed to the last year of records during pretraining. This ensures the model focuses on learning patterns from prior data while preserving temporal integrity for downstream applications. These tasks enable the EHR encoder to effectively capture longitudinal health patterns and contextual relationships inherent in EHR data.

In some cases, the EHR encoder used by the encoding module 222 uses a bidirectional encoder representations from transformers (BERT), such as a Med-BERT architecture, to capture patterns in diagnosis codes within structured health records. In such cases the EHR encoder can be independently pre-trained using the same pretraining tasks as Med-BERT, such as masked code prediction and prolonged length of stay prediction.

Med-BERT is based on the BERT transformer architecture, specifically tailored for encoding sequential EHR data. Med-BERT includes code embeddings, visit embeddings, and attention mechanisms. The code embeddings represent individual diagnosis codes, capturing their semantic and clinical meaning. The visit embeddings represent each patient visit as a unique unit, helping to distinguish events across time. The attention mechanism enables the EHR encoder to learn relationships between diagnosis codes within and across visits. Med-BERT includes masked code prediction for predicting randomly masked diagnosis codes within an EHR sequence, based on their surrounding context. It also includes prolonged length-of-stay prediction for identifying whether a patient experienced a prolonged hospital stay (e.g., >7 days) based on their complete EHR sequence.

Med-BERT is particularly effective for encoding EHR data because it captures complex dependencies between diagnoses and the progression of a patient's health over time. The resulting embeddings preserve both semantic and temporal relationships, making them highly informative for downstream tasks. Embeddings using such encoding can provide structured and contextualized clinical insights, enhancing predictive accuracy and interpretability.

In other cases, the structured EHR data can be encoded using other suitable encoder architectures; for example, Time-Aware Patient EHR Representation (TAPER), CEHR-BERT, BEHRT. Generally, any suitable encoder can be used as long as the encoder is capable of extracting relevant clinical information and incorporating temporal details of patient visits into its latent representations.

At block 306, in some case, the modalities module 226 encodes the received supplementary modality data using a supplementary encoder to produce latent representations of the supplementary modality data. In the case where the supplementary modality data is structured data from a wearable device, such latent representations capture temporal patterns in activity data within the wearable data and generally reflect wearable-derived health patterns. The modalities module 226 uses the encoder to generate a robust time-series encoding suited for the supplementary modality data. In the case of wearable data, such encoding allows for recognizing trends in activity levels, such as periods of high or low activity, that generally correlate to health outcomes (and are thus most predictive of health-related outcomes).

In an example, the supplementary encoder employed by the modalities module 226 is a one-dimensional convolutional neural network (1D-CNN) combined with fully connected layers. In such example, the model can be trained to predict mortality using step sequences-per-minute as training data. Following training, the layer preceding the classification layer can be adjusted dimensional to function as the supplementary encoder, enabling it to capture temporal patterns in activity data. In an example, the training data can include time-stamped step data from a wearable device, with the pretraining task consisting of predicting mortality by identifying patterns in activity levels linked to health outcomes. The loss function can include a weighted binary cross-entropy is used to address class imbalances in mortality data. The feature extraction of the 1D-CNN can capture patterns, such as activity frequency, intensity, and temporal trends; distinguishing between periods of high and low movement. This pretraining enables the 1D-CNN to learn meaningful representations of time-series activity data, which are integrated into the multimodal approach of the system 100. The 1D-CNN is particularly well-suited for encoding activity data because it can effectively capture both localized and general trends in time-series data. Convolutional filters identify localized patterns, such as short bursts of activity or brief sedentary periods, while deeper layers of the network combine these localized features to uncover broader trends over longer time scales. This hierarchical feature extraction allows the CNN to represent activity data in a way that is both detailed and comprehensive. Additionally, 1D-CNNs are computational efficient and can handle sequential, continuous data, making it ideal for processing wearable-derived time-series data.

The 1D-CNN supplementary encoder of the above example outputs latent representations in the form of multi-dimensional vectors that encapsulate key patterns in wearable activity data. These vectors capture temporal trends, such as changes in activity frequency, intensity, and duration over time. By analyzing localized and general trends in the activity data (e.g., step data), the supplementary encoder distills raw, high-dimensional input into a lower-dimensional format. This output retains critical information about activity patterns and their temporal dynamics, ensuring effectiveness for downstream prediction tasks. In further cases, other suitable machine learning models can be used for the supplementary encoder.

In some cases, the modalities module 226 encodes additional supplementary data modalities to generate embedding vectors that capture more nuances in auxiliary contextual data. The encoding of the additional data modalities provides additional flexibility and can provide a more comprehensive understanding of patient health and behaviour. For example, nutritional data can be the additional supplementary data modality. In such example, the input module, for example as part of block 302, also receives nutritional data as a further supplementary data modality. At block 308, the modalities module 226 uses an additional data encoder for the received additional supplementary modality data, such as the nutritional data. In an example, the additional encoder can be different than the supplementary encoder, such as a multi-layer perceptron (MLP) where the supplementary data is nutrition data; such encoder can be specifically designed and pretrained to process structured dietary information. For the case of nutritional data, the encoder encodes patterns in nutritional intake that may influence disease risk, allowing the present invention to learn from dietary behaviours alongside clinical and activity data.

In the case of nutritional data, the MLP, as part of the additional data encoder, can be pretrained using detailed nutritional data, for example, from the U.K. Biobank. Such nutritional data includes both food group consumption and nutrient-level information. Each individual's data is represented as a feature vector with components such as:
Food group consumption: Average daily or weekly intake of fruits, vegetables, grains, dairy, meat, fish, and beverages;
Macronutrients: Energy (calories), protein, fat, and carbohydrate intake, including subcategories like saturated fats and fiber;
Micronutrients: Vitamins and minerals; and
Derived metrics: Ratios like protein-to-calorie and fat-to-calorie, which help capture dietary balance.

The training task of the MLP predicts a health-related outcome using labels of that outcome from the data set; for example, predicting mortality using mortality labels from the dataset. Such model has a loss function that comprises a weighted binary cross-entropy to address class imbalances in the mortality data. Feature extraction of the MLP model can include learning relationships between dietary patterns and mortality risk, identifying key predictors such as nutrient deficiencies or imbalances.

An MLP is particularly useful for encoding nutritional data because it excels at modeling relationships between structured inputs and outputs. It can capture both direct associations (e.g., high sodium intake linked to mortality) and complex interactions (e.g., the interplay of macronutrient ratios). The trained MLP additional encoder outputs embeddings that encapsulate an individual's dietary profile, which can be integrated into an integrated embedding space for multimodal prediction tasks.

In further cases, the supplementary data modality can include other captured activities (for example, heart rate) to be used in association with other ground truth data (for example, predictions of hospitalization rate). It is understood that any suitable health-related modalities can be used for the supplementary data modality; for example, textual data (e.g., clinical notes), demographic data, nutritional data, questionnaire data, lab test data, questionnaire data, electrocardiogram data, electroencephalogram data, image data (e.g., radiology images, pathology slides, or visual health records), and the like. In some cases, the supplementary data modalities can be used instead of the wearable activity data such that the integrated embedding space, described below, includes the latent representation of the EHR data and the latent representation of the other health-related modality.

In further cases, as described with respect to block 308, by using additional encoders, the additional data modalities can be further added by the modalities module 226 to provide further health related modalities to the analysis of the system 100. In such cases, the integrated embedding space, described below, includes latent representations from the EHR data, the supplementary modality data, and one or more further health-related modalities. This modular ability to include further data sources allows the system 100 to capture complex health patterns and further improve the accuracy and personalization of the health-related predictions.

At block 310, the embedding module 230 combines the embeddings determined by the health records encoding module 222 and the modalities module 226 in order to form, or update, an integrated embedding in an integrated embedding space for a health-related predictive LLM model. In some cases, the embedding module 230 replaces the embeddings of designated placeholder tokens with the integrated embedding to create a comprehensive embedding that integrates all relevant inputted data.

The embedding module 230 can combine the separate embeddings into the integrated embedding space by performing at least one of encoding alignment, prompt integration, embedding replacement. In an example, firstly, performing encoding alignment by passing each encoder's embeddings through a fully connected layer to ensure they are transformed into the same dimensional space and appropriately scaled; enabling effective integration. Secondly, performing prompt integration by passing a prompt, which includes textual information and placeholder tokens, through an embedding layer of an LLM model. Thirdly, by performing embedding replacement by replacing embeddings corresponding to the placeholder tokens in the LLM's output with transformed embeddings from each encoder (outputs of the fully connected layers).

The placeholder tokens (e.g., <EHR>, <Activity>) serve as markers within the embedding space and represent where different types of data (such as EHR or activity data) will be inserted. These tokens are replaced with the actual embeddings obtained from the respective encoders (EHR encoder, supplementary encoder, and additional encoders).

At block 312, the prediction module 232 uses the integrated embedding to generate a prediction from a predictive LLM. The prediction can include probabilities of a health-related outcome as prediction output (e.g., prediction of a disease); however, any suitable health-related prediction can be generated. Any suitable LLM can be used, for example, Mistral, Llama, or the like are used. The calculated probabilities can have a variety of suitable forms.

Training, or re-training, of the predictive LLM by the prediction module 232 comprises taking a training dataset comprising data in each of the modalities, integrating the modalities into the integrated embeddings, and training the predictive LLM end-to-end to predict the target health-related outcome (e.g., risk of disease) from the integrated embeddings. The training dataset for training the predictive LLM comprises associated labeled health outcomes (e.g., disease diagnosis, mortality risk, or the like). Training of the predictive LLM can use, for example, weighted binary cross entropy as a loss function. In this way, the predictive LLM can be trained on multimodal datasets that integrate the diverse data modalities; for example, a training dataset that includes EHR, wearable sensor data, nutritional data and other relevant inputs all paired with the labeled health outcomes.

In some cases, the prediction output of the prediction layer may be in the form of a classification output. In an example, the target of the prediction layer can be a binary classification (for example, mortality). The prediction layer computes the logits for each class, i.e., yes and no, which are used to determine a loss during training. In some cases, the logits can be converted into probabilities using a sigmoid function for a classification task. For example, in an experiment conducted by the present embodiments, the model was trained to predict mortality risk as a binary classification.

At block 314, the output module 234 outputs the prediction to the data storage 164, the database 114, the interface module 168 and/or the network module 176.

During inference, the integrated embedding space used to generate the prediction can include the embeddings of just the EHR data, the embeddings of just the supplementary modality data, the embeddings of both the EHR data and the supplementary modality data, the embeddings of the supplementary modality data and the additional supplemental modality data, or the embeddings of all of the EHR data, the supplementary modality data, and the additional supplemental modality data. Whereas during training of the predictive LLM, the integrated embedding space includes the embeddings of both the EHR data and the supplementary modality data, or the embeddings of all of the EHR data, the supplementary modality data, and the additional supplemental modality data

In some cases, output of the predictive LLM can be framed in a binary classification task; particularly, in a way that leverages the LLM's natural language capabilities. The predictive LLM model can be prompted to output “Yes” or “No” as the output, and thus, the task can be simplified into explicit, interpretable tokens that correspond directly to the classification labels (e.g., “Yes” for positive and “No” for negative). Such approach enables the use of logit-based evaluation to calculate the model's confidence for each classification. Specifically, logits represent raw scores for the “Yes” and “No” tokens that are extracted directly from the LLM's output layer. A label assignment can include determining the token with the higher logit, which in turn determines the predicted label. The logits can be normalized (e.g., via sigmoid) to calculate probabilities, enabling performance metrics such as accuracy, precision, recall, and confidence thresholds. Such approach aligns the LLM's natural language outputs with binary classification tasks, while allowing evaluation through logits and probabilities.

In some cases, the predictive LLM can be used to encode instructions and demographic details received as input by the input module 220 because such models inherently understand the context of textual inputs. The embeddings generated by the predictive LLM naturally capture the semantic meaning of such inputs, which are then directly used to make the above Yes/No predictions. Generally, no additional encoder is required for the textual inputs; instead, the pre-trained capabilities of the LLM are leveraged to encode the textual information, and these embeddings are used to generate the prediction once integrated with the latent representations of the other non-textual modalities.

Advantageously, the present embodiments can be adapted to predict the probability of other health-related outcomes, or disease outcomes, by replacing certain labels. As an example, in order to predict the probability of other diseases, the mortality labels can be replaced with labels corresponding to the disease to be predicted. Examples of other diseases that can be predicted includes, but is not limited to, chronic diseases (e.g., COPD, chronic kidney disease), cardiovascular diseases (e.g., heart disease, hypertension), and diabetes. In further cases, the predictive LLM can generate a prediction for health-related outcomes that are not diseases; for example, hospital readmission risk, mental health conditions (e.g., depression risk), health self-assessment, and health related quality of life. Each predictive outcome leverages specific patterns from the integrated data modalities. The system can be adapted to predict multiple diseases and health conditions simultaneously by using a multi-class classification task instead of the binary classification task described above.

The predictive LLM is generally trained end-to-end using disease-specific labels (e.g., heart disease, diabetes) with the combined modalities in the integrated embedding space; thus, optimizing the model for accurate disease prediction. In some cases, the EHR encoder is the same prior to final training regardless of the target health-condition to predict due to its pretraining tasks unrelated to the target. In some cases, pretraining can be performed with a specific health condition to be predicted; however, in many cases, the end-to-end training can incorporate the different modalities directly, without the need for separate pretraining. Training of the predictive LLM ultimately determines how much each modality contributes to predicting a particular target health condition. This allows the predictive LLM model to dynamically adjust the relevance of each modality, optimizing its performance by weighting the importance of each data source for specific disease predictions.

In some cases, the predictive LLM can be trained using a weighted binary cross-entropy loss function, designed to handle class imbalances often present in disease prediction tasks. The loss function can adjust a penalty factor in the model for misclassification by applying higher weights to the minority class, for example to the true disease cases, to encourage the model to learn from both positive and negative cases effectively. In such cases, training proceeds until the loss function converges or substantially converges.

In some cases, AUROC (Area Under the Receiver Operating Characteristic) and F1 scores can be determined during training, in addition to the minimization of loss. These factors can be used as metrics for evaluating model performance in its prediction. This approach helps ensure the predictive machine learning model of the present invention performs well across various metrics as training progresses.

The present inventors conducted example experiments to verify the substantial advantages of the present embodiments. In one such experiment, a large-scale biomedical dataset comprising in-depth genetic and health information from approximately 500,000 individuals across the United Kingdom was used to verify the predictive capabilities of the system 100. This dataset included longitudinal EHR and accelerometer data, enabling comprehensive analysis of both historical health events and real-time movement patterns. EHR data sequences encompassed diagnosis codes across health events, providing a timeline of clinical diagnoses, treatments, and outcomes. In addition to the EHR data, activity data of each individual was represented as a structured time series of minute-by-minute step counts; which was extracted from processing of accelerometer data. In the above-mentioned evaluation, approximately 70000 individuals were identified with both activity and EHR data available meeting the criteria of sufficient of both EHR and activity data. Among these individuals, approximately 3% of individuals had recorded mortality events.

The experimental performance of the multimodal framework was evaluated under three configurations, with results presented in TABLES 1 to 3. To further evaluate the contribution of each data modality, TABLES 4 and 5 present the predictive performance of single-modality encoder models. These results demonstrate the utility and robustness of the system 100 for disease prediction.

TABLE 1

| | | | True | True | False | False |
|---|---|---|---|---|---|---|
| Dataset | Accuracy | AUROC | Positives | Negatives | Positives | Negatives |
| Training | 0.8590 | 0.8945 | 1231 | 40454 | 6531 | 312 |
| Validation | 0.8442 | 0.8334 | 219 | 8560 | 1518 | 102 |
| Test | 0.8476 | 0.8536 | 234 | 8580 | 1490 | 95 |

TABLE 1 shows the experimental performance of the system 100, utilizing a combination of EHR and wearable activity data. The high accuracy and AUROC score values demonstrate strong predictive performance of the system 100, which is particularly indicative of the benefit of combining both EHR and wearable data to capture comprehensive health insights.

TABLE 2

| | | | True | True | False | False |
|---|---|---|---|---|---|---|
| Dataset | Accuracy | AUROC | Positives | Negatives | Positives | Negatives |
| Training | 0.8431 | 0.8895 | 1244 | 39669 | 7316 | 299 |
| Validation | 0.8285 | 0.8265 | 226 | 8390 | 1688 | 95 |
| Test | 0.8291 | 0.8483 | 236 | 8386 | 1684 | 93 |

TABLE 3

| | | | True | True | False | False |
|---|---|---|---|---|---|---|
| Dataset | Accuracy | AUROC | Positives | Negatives | Positives | Negatives |
| Training | 0.5857 | 0.6633 | 994 | 27431 | 19554 | 549 |
| Validation | 0.5876 | 0.6725 | 206 | 5904 | 4174 | 115 |
| Test | 0.5854 | 0.6455 | 205 | 5883 | 4187 | 124 |

TABLE 2 illustrates the example experiments conducted in the case where the activity data was replaced with zeros, effectively isolating the EHR data's contribution. TABLE 3 illustrates the example experiments conducted in the case where the EHR data was replaced with zeros, effectively isolating the activity data's contribution.

The example experiments investigated whether the changes in the predictive model's predictions relative to the EHR-only model aligned with the predictions from the activity-only model. The analysis revealed that 79.8% of cases exhibited this alignment, indicating that the changes in predictions from the full model (both increases and decreases) relative to the EHR-only model corresponded with the activity-only predictions. This suggests that the activity data meaningfully contributes to the adjustments in risk predictions in the full system, either enhancing or attenuating the predicted risk based on the activity patterns. Additionally, it was observed that a moderately strong positive correlation (r=0.771) between the prediction differences (full configuration vs. EHR configuration) and the predictions from the activity-only configuration. This correlation underscores the significant role that activity data plays in enriching the predictive model's predictive capabilities when combined with EHR data.

The example experiments investigated the use of combined EHR-activity data during inference, in addition to isolated EHR data and isolated activity data during inference. The example experiments strongly support that the system can use both, or either, of the modalities at inference time, where the model employed by the system 100 was trained using both modalities, while maintaining its predictive capabilities. A practical advantage of this predictive capability in healthcare is the ability to make accurate predictions when both modalities are not available.

To further evaluate the contribution of each data modality, TABLES 4 and 5 show the predictive performance of single-modality encoder models. These results demonstrate the utility and robustness of the system 100 for disease prediction.

TABLE 4

| | | | True | True | False | False |
|---|---|---|---|---|---|---|
| Dataset | Accuracy | AUROC | Positives | Negatives | Positives | Negatives |
| Training | 0.8420 | 0.8915 | 1238 | 39622 | 7363 | 305 |
| Validation | 0.8292 | 0.8240 | 214 | 8409 | 1669 | 107 |
| Test | 0.8340 | 0.8440 | 237 | 8436 | 1634 | 92 |

TABLE 4 shows the predictive performance of the EHR encoder as an individual single-modality encoder for EHR data. The EHR encoder (using Med-BERT) was evaluated using simple MLP layers on its latent spaces and using the same EHR data that was used for the previous example experiments. The results of the EHR encoder show relatively high accuracy and AUROC, comparable to the full model tested with isolated EHR data.

TABLE 5

| | | | True | True | False | False |
|---|---|---|---|---|---|---|
| Dataset | Accuracy | AUROC | Positives | Negatives | Positives | Negatives |
| Training | 0.6366 | 0.6700 | 732 | 23970 | 13604 | 494 |
| Validation | 0.6390 | 0.6711 | 308 | 10299 | 5776 | 217 |
| Test | 0.6312 | 0.6675 | 260 | 8450 | 4913 | 177 |

TABLE 5 shows the predictive performance of the supplementary encoder as an individual single-modality encoder for wearable activity data. The supplementary encoder was evaluated using the same activity data that was used for the experimental evaluation of the previous example experiments. The performance of the supplementary encoder is comparable to that of the full model when using only activity data. Despite lower accuracy and AUROC factors of the use of isolated activity data, in comparison to the results of isolated EHR data, the results demonstrate the utility of activity data in providing predictive insights, particularly for dynamic health changes.

The results of the example experiments demonstrate the capability of the present invention to offers a fuller view of patient health, and specially its capability to provide a comprehensive and nuanced assessment of disease risks by capturing both long-term health patterns and real-time activity data.

In an example, the system 100 can be used in a scenario when EHR data of a patient shows cardiovascular health issues an, at the same time, the wearable data of the patient indicates low levels of physical activity and irregular sleep patterns. Using the system 100, the EHR encoder encodes the EHR data, while the supplementary encoder encodes the time series data from the wearable. With these two encodings combined, the system 100 predicts a probability of cardiovascular disease. Such prediction can then be inputted to a secondary LLM that can use this determined probability to generate explanations and/or personalized recommendations, such as increasing physical activity and monitoring diet. The secondary LLM can either be the same pretrained LLM described above, a medical-specific LLM, an LLM fine-tuned on general medical context, or a generic LLM, as appropriate. The secondary LLM can be used to interpret the predictions from the system 100 and provide clear, understandable explanations and context to the user, particularly regarding the associated risks.

The example experiments illustrate that the integration of EHR and wearable time-series data provides substantially enhanced disease prediction. Such integration is particularly challenging as the data from EHR and wearable devices have significant differences in nature, type, and source. Additionally, the data can be structured, unstructured, or semi-structured clinical data or continuous time-series data. By combining disparate data modalities, the present invention provides a cohesive approach for generating comprehensive health predictions. Particularly, combining longitudinal data from EHR with real-time physiological parameters obtained from wearable devices. By leveraging both historical and immediate health indicators, the present invention facilitates improved analysis for identifying disease risks and supporting predictive healthcare models.

As illustrated in the example experiments, the present invention employs several distinct encoding models, including an encoder model tailored to the structured nature of EHR data, capturing longitudinal health patterns, and another encoder optimized for wearable data, identifying activity-based trends relevant to disease risk. The latent representations from each encoder are integrated into the integrated embedding space to enhance prediction accuracy. Such approach ensures robustness and adaptability across diverse healthcare scenarios. The output of each encoder can be further refined through additional processing layers to ensure compatibility and scalability. For example, such further refining can include passing each encoder's embeddings through a fully connected layer, which not only changes the dimension of the embeddings but also ensures they are appropriately scaled and aligned. Such additional processing makes sure that all embeddings, regardless of their original size or scale, are compatible with each other. After this adjustment, the embeddings replace the placeholder tokens (e.g., <EHR>, <Activity>), allowing the predictive LLM to combine them seamlessly and integrate all the data for accurate predictions. The integrated embedding space is leveraged to facilitate, for example, disease probability calculations, contextualized decision-making, and adaptable predictions.

The example experiments illustrate that the system 100 uses the integrated embeddings to capture temporal relationships from the wearable data and longitudinal relationships from the EHR data in a single unified feature space, thereby enabling real-time updates to patient health risk. The system 100, thus, can capture the complementary strengths of EHR and wearable data to provide a comprehensive health assessment. While each modality independently contributes predictive value, their combination offers a fuller understanding of patient health, enabling precise and proactive disease risk predictions. In this way, the present inventors discovered that by representing wearable activity patterns in a compressed temporal latent space and merging those with EHR-based embeddings, the model captures cross-modal correlations in a way that prior approaches, such as HeLM, could not.

Advantageously, the present embodiments involve training end-to-end; in contrast to other approaches, such as HeLM, that only train encoders within its system and freezes the LLM. End-to-end training offers at least two significant advantages:
Ability to understand and integrate cross-modal interactions: The LLM learns to model how different modalities (e.g., wearable data and EHR data) interact with each other, capturing both their dynamic relationships and temporal dependencies. For example, temporal patterns in wearable activity may carry different implications depending on contextual information from the EHR, such as recent diagnoses. The same temporal patterns (e.g., witnessing a drop in activity) might indicate different risks based on the patient's EHR history, which the model employed by the system 100 can learn through the joint optimization. This enables the model to holistically interpret how one modality influences or complements another.
Ability to optimize interactions for task-specific predictions: Training the model employed by the system 100 ensures that the learned cross-modal interactions are tailored to a specific prediction task, such as predictions for mortality risk or prediction for specific diseases. The model dynamically adjusts the importance of each modality based on task relevance, allowing it to focus on the most critical features. This enhances both predictive accuracy and relevance.

The system 100 advantageously makes use of specialized encoders that excel at predictions independently. Unlike other approaches, such as HeLM that uses MLP and generic pretrained ResNet+MLP due to simpler modalities, the encoders of the present embodiments can be tailored to capture modality-specific patterns effectively. The specialized encoders act as robust standalone predictors, forming a strong foundation for cross-modal integration. With the specialized encoders, the model employed by the system 100 can effectively correlate modality-specific data, and thus, substantially improving overall model performance.

As evidenced in the example experiments, the system 100 is able to provide predictions for all-cause mortality at a substantially high performance. Additionally, the zero-shot performance of the system 100 is advantageously effectively equivalent to random; which differs from other approaches, such as HeLM, that have greater zero-shot performance (HeLM is approximately 0.61 for all-cause mortality using expanded features).

Effectively integrating a combination of EHR and wearable data modalities into LLM models is non-trivial and represented a substantial challenge due to the need to balance reliance on both data sources. Challenges include the limited dataset availability with EHR and wearable data, as well as the inherent complexity of developing and training with such modalities.

Advantageously, the integration of EHR, activity data, and other available modalities can enable the creation of various other suitable vertical AI-driven agents that operate on top of the multimodal data prediction layer; for example, each designed to address specific aspects of insurance and financial services. These agents can leverage multimodal predictions, including underwriting assistants for precise and dynamic risk assessment, claims agents that streamline and automate claims processing while ensuring fraud resistance, and financial coaching tools that provide personalized, health-linked financial strategies. Additionally, such approach can be advantageously used to facilitates the development of insurance creator agents capable of designing adaptive, real-time insurance products tailored to individual health profiles and ecosystem agents that offer tailored wellness recommendations and product suggestions aligned with users' needs and behaviors. Together, these agents can form a robust, interconnected framework that leverages multimodal health data to enhance personalization, efficiency, and adaptability across the ecosystem. Integrating diverse data modalities enhances the functionality of these agents by offering a holistic understanding of user health and behavior, empowering each agent with contextually rich, multidimensional insights for precise and proactive decision-making.

The model used by the system 100 generally enhances explainability by allowing a focused interpretability analysis of each modality and the overall integration. Through the use of attention mechanisms, an evaluation can be performed for how the model prioritizes features from different modalities, such as EHR and wearable data, in its decision-making. This modality-level interpretability can be further deepened by examining the individual encoders. For the EHR encoder, attention weights can be analyzed to understand the significance of repeated diagnoses or temporal trends within the health records. Similarly, the wearable encoder's feature maps can be studied to identify which time-series patterns, such as periods of inactivity, drive the predictions. By dissecting the attention and feature extraction processes at both the integrated and encoder-specific levels, the system 100 can provide a comprehensive framework for understanding and validating its predictions, aligning with the growing demand for transparent and interpretable AI in critical applications.

In some cases, embodiments of the present disclosure can be implemented on specialized hardware and software platforms designed to handle the extensive computational load, thereby offering significant improvements over conventional human-performed tasks. In this way, the interaction between these components/steps and the technical outcomes achieved by the present embodiments, such as enhanced predictive accuracy, are impractical for human cognition to replicate due to the scale and complexity of the operations involved. In some cases, there is increased reliability and robustness due to integrated multimodal processing, which leverages machine learning models that adapt and optimize in ways beyond human cognitive capabilities.

Although the foregoing has been described with reference to certain specific embodiments, various modifications thereto will be apparent to those skilled in the art without departing from the spirit and scope of the invention as outlined in the appended claims. The scope of the claims should not be limited by the preferred or example embodiments but should be given the broadest reasonable interpretation consistent with the specification as a whole.

1. A computer-implemented method for predicting a health-related outcome, the method comprising:
receiving electronic health records data;
generating a first set of latent representations using a first trained encoder, the first trained encoder taking the electronic health records data as input, the first trained encoder trained to extract health patterns from the inputted electronic health records data;
generating integrated embeddings by integrating the first latent representations into an integrated embedding space;
generating a prediction for the health-related outcome using a trained large language model, the trained large language model taking the integrated embeddings in the integrated embedding space as input, a training dataset for training of the large language model comprises electronic health records data with associated labelled health outcomes and supplementary modality data with associated labelled health outcomes, the integrated embeddings during training of the large language model are generated by integrating first latent representations generated by the first trained encoder from the electronic health records data with embeddings of a second set of latent representations generated by a second trained encoder from the supplementary modality data, wherein the integrating of the first latent representations and the second latent representations comprises (i) performing encoding alignment to transform into a same dimensional space and to scale the first and second latent representations relative to each other, and (ii) performing embedding replacement by replacing placeholder tokens in the integrated embedding space with the transformed embeddings so that non-textual data is treated as token-level input;
outputting the prediction for the health-related outcome.

2. The method of claim 1, wherein the supplementary modality data comprises time-series wearable activity data.

3. The method of claim 1, wherein the health patterns extracted by the first trained encoder comprises patterns in temporal sequences of diagnosis codes.

4. The method of claim 1, wherein the first trained encoder is trained using masked code prediction by predicting randomly masked diagnosis codes based on surrounding diagnosis codes.

5. The method of claim 4, wherein the first trained encoder uses bidirectional encoder representations from transformers.

6. The method of claim 1, wherein the second trained encoder comprises a one-dimensional convolutional neural network combined with fully connected layers.

7. The method of claim 6, wherein the training data for the second trained encoder comprises time-stamped activity data, and wherein during training of the second trained encoder, the second trained encoder is trained to predict mortality by identifying patterns in activity levels linked to health outcomes.

8. The method of claim 1, wherein the training dataset further comprises additional supplementary modality data, and wherein the integrated embeddings further comprise integrated embeddings of a third set of latent representations generated by a third trained encoder from the additional supplementary modality data.

9. The method of claim 8, wherein the additional supplementary modality data comprises nutritional data, and wherein the third trained encoder comprises a multi-layer perceptron.

10. (canceled)

11. A system for predicting a health-related outcome, the system comprising a processing unit in communication with a data storage, the data storage comprising instructions for the processing unit to execute:
an input module to receive electronic health records data;
a health records encoding module to generate a first set of latent representations using a first trained encoder, the first trained encoder taking the electronic health records data as input, the first trained encoder trained to extract health patterns from the inputted electronic health records data;
an embedding module to generate integrated embeddings by integrating the first latent representations into an integrated embedding space;
a prediction module to generate a prediction for the health-related outcome using a trained large language model, the trained large language model taking the integrated embeddings in the integrated embedding space as input, a training dataset for training of the large language model comprises electronic health records data with associated labelled health outcomes and supplementary modality data with associated labelled health outcomes, the integrated embeddings during training of the large language model are generated by integrating first latent representations generated by the first trained encoder from the electronic health records data with embeddings of a second set of latent representations generated by a second trained encoder from the supplementary modality data, wherein the integrating of the first latent representations and the second latent representations comprises (i) performing encoding alignment to transform into a same dimensional space and to scale the first and second latent representations relative to each other, and (ii) performing embedding replacement by replacing placeholder tokens in the integrated embedding space with the transformed embeddings so that non-textual data is treated as token-level input;
an output module to output the prediction for the health-related outcome.

12. The system of claim 11, wherein the supplementary modality data comprises time-series wearable activity data.

13. The system of claim 11, wherein the health patterns extracted by the first trained encoder comprises patterns in temporal sequences of diagnosis codes.

14. The system of claim 11, wherein the first trained encoder is trained using masked code prediction by predicting randomly masked diagnosis codes based on surrounding diagnosis codes.

15. The system of claim 14, wherein the first trained encoder uses bidirectional encoder representations from transformers.

16. The system of claim 11, wherein the second trained encoder comprises a one-dimensional convolutional neural network combined with fully connected layers.

17. The system of claim 16, wherein the training data for the second trained encoder comprises time-stamped activity data, and wherein during training of the second trained encoder, the second trained encoder is trained to predict mortality by identifying patterns in activity levels linked to health outcomes.

18. The system of claim 11, wherein the training dataset further comprises additional supplementary modality data, and wherein the integrated embeddings further comprise integrated embeddings of a third set of latent representations generated by a third trained encoder from the additional supplementary modality data.

19. The system of claim 18, wherein the additional supplementary modality data comprises nutritional data, and wherein the third trained encoder comprises a multi-layer perceptron.

20. (canceled)
